(19) United States
(12) Patent Application Publication
(10) Pub. No.: US 2023/0218827 A1
(43) Pub. Date: Jul. 13, 2023

(54) AUTOINJECTOR WITH DISCHARGE STOP

(71) Applicant: Ypsomed AG, Burgdorf (CH)

(72) Inventors: Simon Scheurer, Bern (CH); Markus Tschirren, Burgdorf (CH); Leos Urbanek, Bern (CH); Gabriel Kalbermatter, Burgdorf (CH); Boris Budesa, Schliern (CH); Jürg Hirschel, Bern (CH); Nicolas Grünig, Courtepin (CH)

(21) Appl. No.: 18/185,057

(22) Filed: Mar. 16, 2023

Related U.S. Application Data
(63) Continuation of application No. PCT/EP2021/076923, filed on Sep. 30, 2021.

(30) Foreign Application Priority Data
Sep. 30, 2020 (CH) 01236/20

(51) Int. Cl. A61M 5/20; A61M 5/32

(52) U.S. Cl. CPC A61M 5/2033; A61M 5/32; A61M 2005/2013

(57) ABSTRACT

An autoinjector having a housing for receiving a product container, a propelling member, a drive for automatically discharging a liquid product, a needle-guard spring for pretensioning a needle-guard sleeve, a grid with a multiplicity of latch elements arranged along the longitudinal direction, and an engagement element which, by engagement in the grid, blocks a discharging movement of the propelling member. A control element of the autoinjector interacts with the engagement element via a control cam such that, when the control element moves in the distal direction by the needle-guard spring, the engagement element is brought into engagement with the grid. Since the force of the needle-guard spring is used for the engagement, the engagement element can be mounted without specific elastic force acting in an engagement direction, and can also be mounted in an articulated manner and/or in a manner limited to pure guidance in the engagement direction.

### CROSS-REFERENCE TO RELATED APPLICATIONS

This application claims is a continuation of International Patent Application No. PCT/EP2021/076923 filed on Sep. 30, 2021, entitled “AUTOINJECTOR WITH DISCHARGE STOP,” which in turn claims priority to Swiss Patent Application No. CH 01236/20 filed on Sep. 30, 2020, entitled “AUTOINJECTOR WITH DISCHARGE STOP,” each of which is incorporated by reference herein, in the entirety and for all purposes.

### TECHNICAL FIELD

The present invention relates to the field of medical injection devices for administering liquid substances, in particular medicaments or medical substances, such as insulin and hormone preparations. The invention relates to an autoinjector having a discharge stop for interrupting a discharging operation.

### BACKGROUND OF THE INVENTION

Injection devices or injection apparatuses for the simplified administration of a substance include, inter alia, so-called autoinjectors which have an energy store element or drive element with which the discharge process can be carried out automatically, i.e., without a force to be supplied or exerted externally by a user. The energy store element or drive element advantageously stores the energy required for an automatic substance dispensing in mechanical form. Such an energy store element or drive element can be a spring which is installed in a tensioned state in the injection device and delivers energy when relaxed. The energy is delivered to a piston rod or a pressure element, which pushes a piston into a product container. The energy store element or drive element may also be provided in order to automate the process of inserting an injection needle. Alternatively, a further separate drive element can be provided for this purpose, or the piercing process takes place manually, i.e., exclusively by a user, without energy stored for this purpose in the injection device being used.

The injection device may comprise a product container holder for accommodating a product container, wherein the product container can be held in the product container holder radially, axially, and preferably also in a rotationally fixed manner. The product container holder may be connected to the housing of the injection apparatus in an axially and rotationally fixed manner or may be movable relative to the housing during an insertion and/or needle retraction process. The product container may be a cartridge for the repeatedly detachable connection to disposable injection needles or a disposable prefilled syringe with an injection needle non-detachably connected thereto. The product container has a hollow cylindrical product container portion which displaceably mounts a piston or plunger. The piston can form a sealing gap with the inner circumference of the product container portion and can be displaced in a distal direction by means of a piston rod in order to dispense product from the product container via the injection needle.

The injection device may have a needle-guard sleeve which, after injection has taken place, projects distally beyond the distal end of the injection needle or is displaced relative to the housing into this needle-guard position while relaxing a needle-guard sleeve spring, in order to prevent accidental access to the injection needle and to thereby reduce the risk of injury. In an autoinjector, the needle-guard sleeve can also serve as a trigger element for triggering the product discharge process, wherein the needle-guard sleeve is displaced relative to the housing in the proximal direction for this purpose. Alternatively, the triggering of the autoinjector can be achieved by actuating a trigger button of the autoinjector, wherein the needle-guard sleeve serves at least as a visual protection before the autoinjector is used.

Patent application WO 2016/205963 A1 describes an exemplary autoinjector comprising a housing with a longitudinal axis, a triggering device and a product container arranged axially fixedly in the housing. The autoinjector furthermore comprises a needle-guard sleeve which is displaceable in a longitudinal direction between a proximal and a distal position and is coupled to a needle-guard sleeve spring as a separate drive element. A first feedback device with a first stop element accelerated by the discharge spring signals the start of the substance dispensing. A second feedback device with a second stop element accelerated toward a stop by the needle-guard sleeve spring serves to generate an acoustic signal after a certain amount of substance has been dispensed. A spiral spring or mainspring in which energy for the automatic discharge of product can be stored is coupled to the triggering device, wherein a first end of the spiral spring is connected to the housing and a second end of the spiral spring is connected in a rotationally fixed manner to a rotating member in the form of a threaded rod arranged coaxially with the longitudinal axis. The threaded rod engages via a thread in a propelling member in the form of a sleeve-like piston rod, which propelling member is not rotating in the housing and which piston rod moves the plunger of the product container at an at least approximately constant discharge rate in the distal direction during a displacement. The autoinjector is designed for prefilled syringes comprising a product container having a predetermined size and a needle, which needle is surrounded prior to use by an elastic needle-guard element and a fixed needle-guard cap or a rigid needle shield RNS to ensure sterility and integrity.

Patent application WO 2015/107180 A1 describes an autoinjector with a discharge stop for interrupting the product discharge if the autoinjector is moved away from the injection site prematurely, i.e., before the entire contents of the product container have been dispensed. The mechanism comprises a grid and an engagement means which is mounted elastically and is held out of engagement with the grid by a needle-guard sleeve in a proximal position. As soon as the needle-guard sleeve is moved distally from the proximal position, the pretensioned engagement element moves into engagement with the grid and thereby interrupts the discharging. The elastic mounting and the shape of the engagement element must be dimensioned as plastic injection-molded parts, such that the force of the discharge spring cannot push the engagement element out of engagement with the grid even without additional securing, and the engagement by a proximally directed force of the needle-guard sleeve can still be released again for continuation of the discharging. Alternatively, the mechanism comprises flexible arms on the needle-guard sleeve, which clamp the piston rod flat as long as the needle-guard sleeve is not in the proximal position. By inserting the needle-guard sleeve, the clamping seat is released and the piston rod can move in the discharging direction.

The term “product,” “medicament,” or “medical substance” in the present context includes any flowable medical formulation which is suitable for controlled administration by means of a cannula or hollow needle in subcutaneous or intramuscular tissue, for example a liquid, a solution, a gel, or a fine suspension containing one or more medicinal active ingredients. A medicament can thus be a composition with a single active ingredient or a premixed or co-formulated composition with a plurality of active ingredients from a single container. The term includes in particular drugs, such as peptides (e.g., insulins, insulin-containing medicaments, GLP-1-containing preparations as well as derived or analogous preparations), proteins and hormones, biologically obtained or active ingredients, active ingredients based on hormones or genes, nutrient formulations, enzymes, and other substances both in solid (suspended) or liquid form. The term also includes polysaccharides, vaccines, DNA or RNA or oligonucleotides, antibodies or parts of antibodies as well as suitable base substances, excipients, and carrier substances.

The term “distal” refers to a side or direction directed toward the front, piercing-side end of the administration apparatus or toward the tip of the injection needle. In contrast, the term “proximal” refers to a side or direction directed toward the rear end of the administration apparatus that is opposite the piercing-side end.

In the present description, the term “injection system” or “injector” is understood to mean an apparatus in which the injection needle is removed from the tissue after a controlled amount of the medical substance has been dispensed. In contrast to an infusion system, the injection needle in an injection system or in an injector thus does not remain in the tissue for a longer period of several hours.

### DESCRIPTION OF THE INVENTION

It is an object of the invention to specify an autoinjector which can reliably interrupt a discharging in the event of premature removal of the autoinjector from the injection site. The object is achieved by a first and a second autoinjector having the features of the independent claims. Preferred embodiments of the invention are the subject matter of the dependent claims.

According to the invention, a first autoinjector comprises a housing defining a longitudinal direction and suitable for receiving a product container with an injection needle at a distal end of the product container. The autoinjector comprises a drive with an energy store in the form of a pretensioned discharging spring for driving a propelling or discharge member in the form of a propelling sleeve in the longitudinal direction for the one-time, automatic discharging of at least a portion of a liquid product contained in the product container through the injection needle. The autoinjector comprises a needle-guard sleeve and a needle-guard spring for pretensioning the needle-guard sleeve in the distal direction, wherein, when the autoinjector is pressed against an injection site, the needle-guard sleeve executes an actuating movement, in particular a triggering or release movement, in the proximal direction, and, when the autoinjector is removed from the injection site, the needle-guard sleeve executes a needle-guard movement in the distal direction. The autoinjector comprises a grid with a multiplicity of rigid locking elements or teeth, as well as a movably mounted engagement element which is adapted to the latching elements and which can block the discharging movement of the propelling member by engaging in a latching element. When the autoinjector is removed from the injection site after only partially discharging, i.e., before a piston impinges on the distal end of the product container, the engagement element is pushed into engagement with the grid by the force of a pretensioned spring.

As a result of the force of a spring being used for the engagement, a mounting of the engagement element itself without a specific elastic force effect or pretensioning can be formed in the engagement direction; in particular, the mounting can also be designed in an articulated manner and/or be limited to a pure guide in the engagement direction. A spring designed as a metallic coil spring also has more easily dimensioned and less aging-dependent elastic properties than a mounting or suspension of the engagement element made of plastic. The blocking of the propelling member in the event of an interruption of the injection, and thus the avoidance of undesired and unpleasant product discharge next to the injection site, are thus reliably ensured. This blocking is particularly useful for autoinjectors having larger volumes of more than 3 ml, in which, in the event of an injection interruption, a considerable amount of liquid can still be dispensed next to the injection site.

According to a preferred embodiment, the drive comprises a rotating drive element in the form of a threaded rod for moving the propelling member in the form of a propelling sleeve with an axial guide element for an exclusively linear propelling movement in the housing. At least two latching elements are arranged on the rotating drive element, distributed concentrically and symmetrically about an axis of rotation over a circumference. In an axial coupling stroke, the engagement element blocks the drive element directly, i.e., not via a transmission or thread via the propelling member or via a further component that is rotatable relative to the drive element.

In a preferred embodiment, the engagement element is released by the needle-guard movement of the needle-guard sleeve for the coupling stroke. In particular, a switching and/or locking sleeve is also moved in the distal direction with the needle-guard movement, whereby cams which interact with the engagement element are released from an initial engagement with a mechanism holder that is fixed to the housing.

In a first advantageous variant, the engagement element is moved by the needle-guard spring during the coupling stroke. Preferably, the latching elements and the engagement element have corresponding coupling surfaces which are designed not parallel or perpendicular to the longitudinal axis, but in each case in the form of oblique, gear-like guide surfaces. When the drive element is at least partially coupled via a latching element, a force is exerted on the engagement element in the proximal direction by the torque of the drive element.

In a second advantageous variant, the engagement element is part of a coupling which, as a result of the actuating movement of the needle-guard sleeve, enables the drive element to rotate directly, i.e., not via a thread and/or a further component that is rotatable relative to the drive element.

In a preferred development, the coupling comprises an axially movable blocking unit with a first coupling element, which element can be removed by an axial triggering stroke or decoupling stroke from a second coupling element for triggering the rotation. The coupling comprises a third coupling element as an engagement element, which can engage in a fourth coupling element via a coupling surface for blocking the rotation of the drive element via an axial coupling stroke.

The second and the fourth coupling element are preferably provided on an extension of the spring coil that is non-rotatably connected to the drive element. Further preferably, the first and the third coupling elements and the second and the fourth coupling elements are identical. The coupling stroke is then set in opposition to the triggering stroke. Alternatively, the first and the third coupling elements are identical and the second and fourth coupling elements are spaced apart axially by distance equal to the sum of the triggering and coupling stroke. The triggering stroke and the coupling stroke take place in the same direction and can thus also be of different magnitudes.

In a third advantageous variant, a first coupling element can be removed by a distal triggering stroke from a second coupling element to trigger the rotation. A third coupling element, different from the first, for blocking the rotation of the drive element, can engage in a fourth coupling element by a proximal coupling stroke via a coupling surface. The first coupling element is preferably arranged on a locking sleeve, while the third coupling element is assigned to a blocking unit.

In a fourth advantageous variant, the engagement element is moved distally by a specific, previously pretensioned coupling spring without further function during the coupling stroke. When the autoinjector is removed from the puncture site, a blocking unit is pushed by the coupling spring to the extent of an axial coupling stroke in the distal direction and is coupled directly to the drive component, so that the said drive component can no longer rotate and the propelling of the propelling element is stopped. Preferably, the blocking unit is moved beforehand when the autoinjector is set in place to the extent of a decoupling stroke in the proximal direction and decoupled from the drive component, as a result of which the discharging is started. In the case of an axially movable threaded rod as a pushing or screwing propelling element, a combination or coexistence of thread and grid on the outer side of the propelling member is somewhat cumbersome, instead of which the blocking of a rotational drive of the axially movable threaded rod is in any case preferred.

In a preferred embodiment of the fourth variant, a coupling comprises a first coupling element or engagement element, for example in the form of a radial projection, which engages in a second coupling element via an axial coupling surface. This engagement is releasable by an axial decoupling stroke of the two coupling elements to release the rotation and can be ensured or compelled to lock the rotation.

In a preferred embodiment of the fourth variant, the coupling comprises an axially movable blocking unit or a coupling sleeve with the first coupling element and an extension of the spring coil with the second coupling element that is non-rotatably connected to the drive element.

In a preferred further development of the fourth variant, the blocking unit is pretensioned in the engagement distally by a coupling spring, wherein the blocking unit is pushed out of engagement in the proximal direction against the coupling spring by the actuating movement via contact with the switching sleeve.

In advantageous embodiments of the fourth variant, a locking sleeve coupled to the needle-guard sleeve is moved by a first partial stroke of the actuating movement, and a cam which is flexibly attached to the blocking unit is released from an axially fixed recess, and the blocking unit is released for a proximal movement. Subsequently, the blocking unit is pushed in the proximal direction into a release position by a second partial stroke of the actuating movement.

According to a preferred embodiment, the autoinjector comprises a grid in the form of a toothed rack having a multiplicity of rigid latching elements or teeth arranged along the longitudinal direction, as well as a flexible engagement element, which is in particular elastically mounted or articulated and adapted to the latching elements. The engagement element can block a discharging movement of the propelling member by means of an—in particular radial or tangential—engagement in the grid which is not exclusively in the longitudinal direction and preferably transverse or perpendicular to the longitudinal direction. For this purpose, the engagement element interacts with a control element via a control cam and, in particular, via a control surface which is inclined relative to the longitudinal axis, said control element being designed and arranged such that, in the case of a movement of the control element driven by the relaxing needle-guard spring in the distal direction, the engagement element is pushed into engagement with the grid.

Preferably, the grid is connected axially fixedly to the propelling member and in particular is arranged on the propelling member. As a result, the stroke or the path of the control element for carrying out the engagement movement is constant and independent of the position of the propelling member at the moment of injection interruption. Alternatively, the grid is fixed axially in the housing and the engagement element is movable, wherein the stroke of the control element depends on the position of the engagement element.

In a further preferred embodiment, the movement of the relaxing needle-guard spring when the autoinjector is removed from the puncture site moves a locking surface of a locking element into a locking position next to the engagement element in the engaged state. The locking element is held in the locking position by the force of the not completely relaxed needle-guard spring and the engagement member is secured in engagement with the latching elements and against a radial or tangential movement. Preferably, the locking element is formed integrally with the control element and the locking surface is a continuation of the control cam in the proximal direction. If the blocking of the propelling element is to be released again by the engagement element, or for the first time before the start of the discharge, the locking element can be moved in the proximal direction against the force of the needle-guard spring by pressing in the needle-guard sleeve.

In an advantageous embodiment, the locking element is held non-detachably in the locking position for the user. Accordingly, the blocking of the propelling element is also irreversibly secured, and the autoinjector is in particular not designed to completely discharge the product after a blocking according to the invention of the propelling member. A further use after a premature removal of the autoinjector is not provided, but the blocking of the propelling member makes it possible to at least determine how much product has actually been injected or remains in the product container. A corresponding value can be transmitted by dedicated electronics or by the user himself/herself to a treating physician and evaluated by said physician for the appropriate reaction to premature termination of the injection.

In an advantageous embodiment, the autoinjector comprises a locking mechanism for locking the needle-guard sleeve in a needle-guard position surrounding the injection needle and into which the needle-guard sleeve is moved out of an intermediate or puncture position by the needle-guard spring when the autoinjector is removed from the injection site. In the needle-guard position, the needle-guard sleeve is coupled in an axially fixed manner to the locking element at least in the proximal direction and is preferably formed integrally therewith. Advantageously, a switching sleeve coupled axially to the needle-guard sleeve takes on the function of the control element and the locking element. A locking member of the locking mechanism is activated in the proximal intermediate position of the needle-guard sleeve at the beginning of the discharging. As an alternative or complementary to a locking of the needle-guard sleeve, the engagement element and the latching elements are designed or shaped such that the engagement can no longer be released by the user, for example by an attractive positive locking of the contact surfaces of the engagement and latching element in which the elements are held by the force of the discharge spring.

In further preferred variants, the engagement element is elastically formed or attached and contacts the latching elements during a discharging movement. As a result, a clicking or a rattling noise is generated, signaling to the user the progressive discharging. As a result, a separate acoustic start and/or end signal can be omitted. The latching elements are preferably depressions or recesses in the outer casing of the propelling sleeve, but no openings or holes through which the engagement element could come into contact with the discharge spring. A distance between the latching elements can be constant or can be reduced in accordance with a decrease in the spring force of the discharge spring during the discharging, so that the clicking noises still sound at regular intervals.

In further advantageous variants, the propelling member is blocked by the locking surface in an initial delivery state of the autoinjector. To this end, the locking surface prevents a retaining cam from getting out of engagement with a recess of the propelling member that is different from the latching elements. The retaining cam can comprise the engagement element as a radially inwardly pointing formation.

Advantageously, the product container or the prefilled syringe is held axially fixedly and not displaceably in the autoinjector. The needle-guard sleeve is preferably a triggering sleeve which, from the initial position or delivery position, triggers the discharging directly by a displacement into the proximal intermediate position or at least enables it to be triggered in the sense of a necessary condition. In the latter case, for example, a trigger button must be actuated for the start of the triggering. An alternative autoinjector with syringe movement comprises a syringe holder for receiving the product container and a syringe movement mechanism for moving the syringe holder and syringe at least in the distal direction. In this case, the engagement member may be attached to the syringe holder. In the case of autoinjectors without syringe retraction into the housing, after the autoinjector has been lifted off the puncture site, the needle-guard sleeve is driven by the needle-guard spring into a final, distal needle-guard position, in which only the needle-guard sleeve laterally shields the needle tip.

### FIGURES

Preferred embodiments of the invention are described below in connection with the appended figures. These embodiments are intended to show basic possibilities of the invention and are in no way to be interpreted as limiting. In the drawings:

FIG. 1 shows an exploded view of a first embodiment of an autoinjector;

FIG. 2 shows a longitudinal section through the autoinjector at the moment the discharging is triggered;

FIG. 3a and FIG. 3b show two longitudinal sections through the autoinjector during the discharging process;

FIG. 4a and FIG. 4b show the two longitudinal sections from FIG. 3a and FIG. 3b in the blocking and locking state;

FIG. 5a and FIG. 5b show two longitudinal sections of a second embodiment in the delivery state of the autoinjector;

FIG. 6 shows a cross section through the autoinjector of the second embodiment before the discharging;

FIG. 7a and FIG. 7b show two longitudinal sections of the second embodiment in the triggered state and in the blocked state;

FIG. 8a, FIG. 8b, and FIG. 8c show three successive states of a third embodiment of an autoinjector;

FIG. 9a and FIG. 9b show two partial longitudinal sections of a fourth embodiment in the delivery state of the autoinjector;

FIG. 10a and FIG. 10b show the longitudinal sections from FIG. 9a and FIG. 9b in the blocked state of the autoinjector,

FIG. 11a and FIG. 11b show two cross sections of the fourth embodiment;

FIG. 12a and FIG. 12b show a partial longitudinal section next to the longitudinal axis in the delivery and blocked state;

FIG. 13a and FIG. 13b show two partial longitudinal sections of a fifth embodiment in the delivery state; and

FIG. 14a and FIG. 14b show two partial longitudinal sections next to the longitudinal axis of the fifth embodiment.

### DESCRIPTION OF THE FIGURES

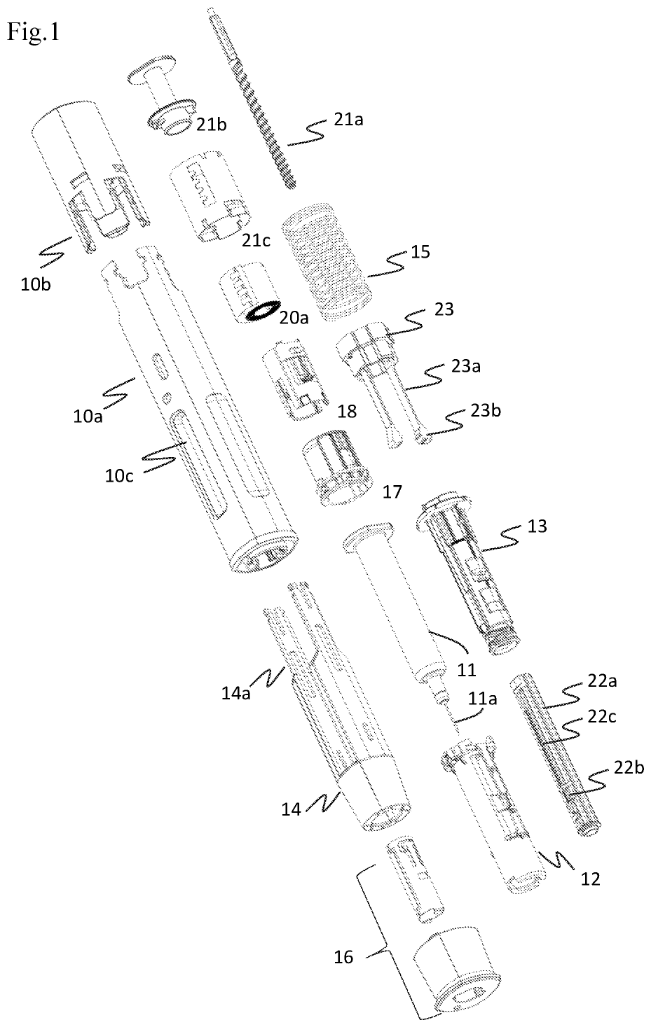

FIG. 1 is an exploded view of the components of an autoinjector according to a first variant of the invention. The autoinjector has a sleeve-shaped, elongated housing with a longitudinal axis L and comprising a distal housing part 10a and a proximal closure cap or end cap 10b which is non-detachably snap-fitted therewith. A product container in the form of a prefilled syringe 11 with an injection needle 11a which is non-detachably fastened to the product container is held in a syringe holder 12, wherein the syringe holder is accommodated in the distal housing part 10a in an axially and rotationally fixed manner. The prefilled syringe 11 is pressed in the distal direction into engagement with a shoulder of the syringe holder 12 by a retaining spring portion of a mechanism holder 13 fixedly anchored in the closure cap 10b. In relation to the housing part 10a, the prefilled syringe 11 is arranged such that the tip of the injection needle 11 a projects beyond the distal end of the housing part 10a by a length corresponding to the subcutaneous or intramuscular piercing depth and is at least laterally protected or covered by a needle-guard sleeve 14 before and after the injection. When the injection needle 11a is inserted into the injection site along the longitudinal axis L, the needle-guard sleeve 14 is pushed in the proximal direction by an actuation stroke and against the force of a needle-guard spring 15 and thereby triggers a product discharge. For this purpose, the needle-guard sleeve comprises two sleeve arms 14a, which are arranged offset or rotated by 90° about the longitudinal axis L with respect to two recesses 10c of the housing designated as viewing windows. After the injection has taken place or when the autoinjector is prematurely removed from the injection site, the needle-guard sleeve 14 can be displaced relative to the housing 10a from the intermediate position along the longitudinal axis L in the distal direction into a needle-guard position and can be blocked there against being pushed back again. The needle-guard spring 15 is a spring made of metal which acts as a compression spring and is designed as a coil spring and acts directly or via a control element or a switching sleeve on the proximal end of the needle-guard sleeve. A proximal end of the needle-guard spring 15 is axially fixed relative to the housing.

A spring package comprises a spiral spring 20a as a discharge spring, a spring coil 21b, and a spring sleeve 21c. The outer end of the spiral spring 20a is anchored in a rotationally fixed manner to the spring sleeve 21c, which in turn is accommodated in a rotationally fixed manner in the housing 10a. The inner end of the spiral spring 20a is connected to the spring coil 21b in a rotationally fixed manner. The spring coil 21b comprises a spring shaft and a distal and a proximal spring flange, which axially delimit the spring volume. The spring package can be mounted as an independent component in the housing of the autoinjector in a completely pretensioned manner and can accommodate coil springs of different widths.

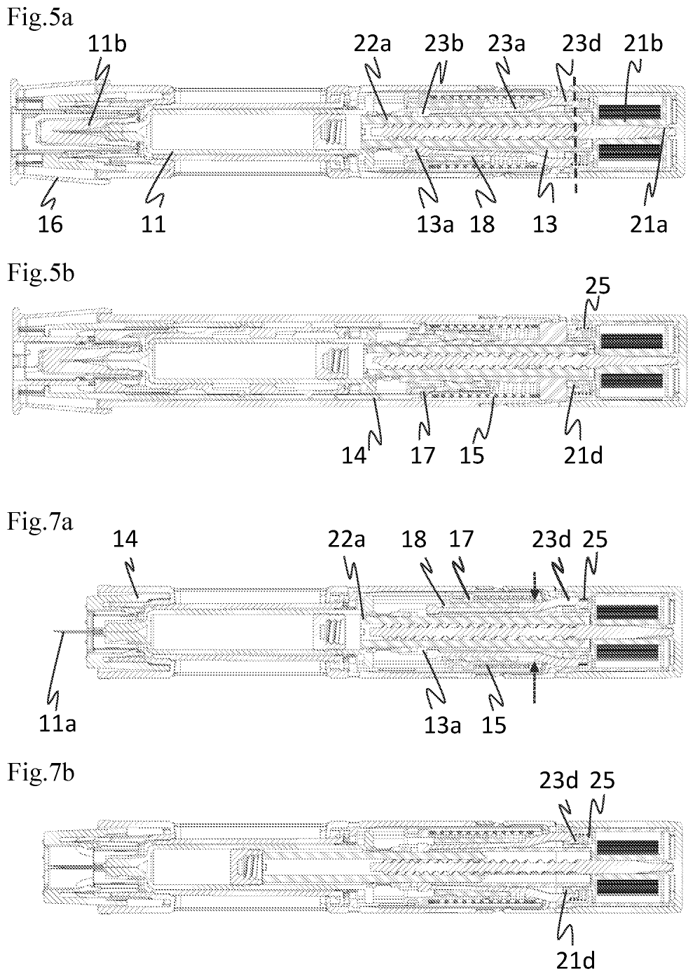

The prefilled syringe 11 comprises a cylindrical syringe body as a product container, at the distal end of which a hollow injection needle 11a is fixedly connected to a syringe shoulder. The injection needle of the prefilled syringe is covered by a needle-guard cap 11b which can be seen in FIG. 5 and is designed as a so-called rigid needle shield (RNS) and comprises a rubber-elastic needle-guard element and a sleeve made of hard plastic. The needle-guard cap protects the injection needle against mechanical effects and contamination, and keeps the injection needle and the product sterile. At the distal end of the autoinjector, in the initial or delivery state thereof, a device cap or pull-off cap 16 is arranged which is axially pulled off and/or twisted off and completely removed along with the needle-guard cap before the autoinjector is used. The syringe holder 12 comprises two fingers, which are fastened at their proximal ends to a holder sleeve of the syringe holder and each have, at their distal ends, an axial support element for the syringe shoulder.

To carry out the discharge, the spiral spring 20a or the spring coil 21b rotates a rotational member in the form of a threaded rod 21a having an external thread, which thread extends at least over a length corresponding to the discharge stroke. The threaded rod 21a is coupled to the spring coil 21b in a rotationally fixed manner or even integrally formed therewith. A propelling element in the form of a propelling sleeve 22a has, at a proximal end on an inner side, a threaded element for engagement in the external thread, comprising a threaded portion with preferably fewer windings than the external thread, or a threaded segment with an extension in the direction of rotation of less than one winding, preferably less than half a winding. The propelling sleeve 22a in the mechanism holder 13 or in the housing is secured against rotation by an axial groove or another deviation from a rotationally symmetrical outer side, so that the rotation generated by the spiral spring 20a is converted into a linear propelling movement. On opposite longitudinal sides, the propelling sleeve 22a has two recesses 22b or openings, each with at least one distally directed edge or blocking surface, and in the proximal direction connecting thereto a grid in each case.

A blocking unit 23 has a sleeve-shaped proximal base with two flexible arms 23a mounted in the distal direction and at each end of which there is a cam 23b. An inner side of the cams 23b is adapted to the recess 22b of the propelling sleeve 22a and, in the delivery state of the autoinjector, blocks an axial movement of the propelling member by an initial engagement in the recess 22b. An engagement element 23c shown in FIG. 3 in the form of a tooth for engagement in the grid is present inside the cams 23b.

A switching sleeve 17 is arranged between a proximal end of the sleeve arms 14a of the needle-guard sleeve 14 and the needle-guard spring 15 and is at least partially surrounded by the needle-guard spring 15. The switching sleeve 17 is preferably snap-fitted or even integrally formed with the proximal end of the sleeve arms 14a. Within and coaxially with the switching sleeve 17, a locking sleeve 18 is arranged with two saw-tooth-shaped locking members 18a, which are shown in FIG. 3 and are spring-mounted on a spring arm pointing in the distal direction. The locking sleeve 18 is coupled to the switching sleeve 17 via the locking members 18a in such a way that an actuating movement of the needle-guard sleeve 14 and the switching sleeve 17 also moves the locking sleeve 18 in the proximal direction. In a proximal end position, the locking members 18a are released by the switching sleeve 17 for an inward movement. Due to the spring effect of the spring arms, the locking members 18a each engage behind a proximally directed edge of the autoinjector or latch into an axially fixed recess of the autoinjector and thus arrest the locking sleeve 18 against a distal movement. When the autoinjector is removed from the puncture site, the switching sleeve 17 is pushed by the needle-guard spring 15 in the distal direction over the locking members 18a, whereupon, as a result of the spring effect of the spring arms, the locking members each engage behind a proximally directed edge of the switching sleeve 17 in a locking position and lock the switching sleeve and the needle-guard sleeve against renewed movement in the proximal direction.

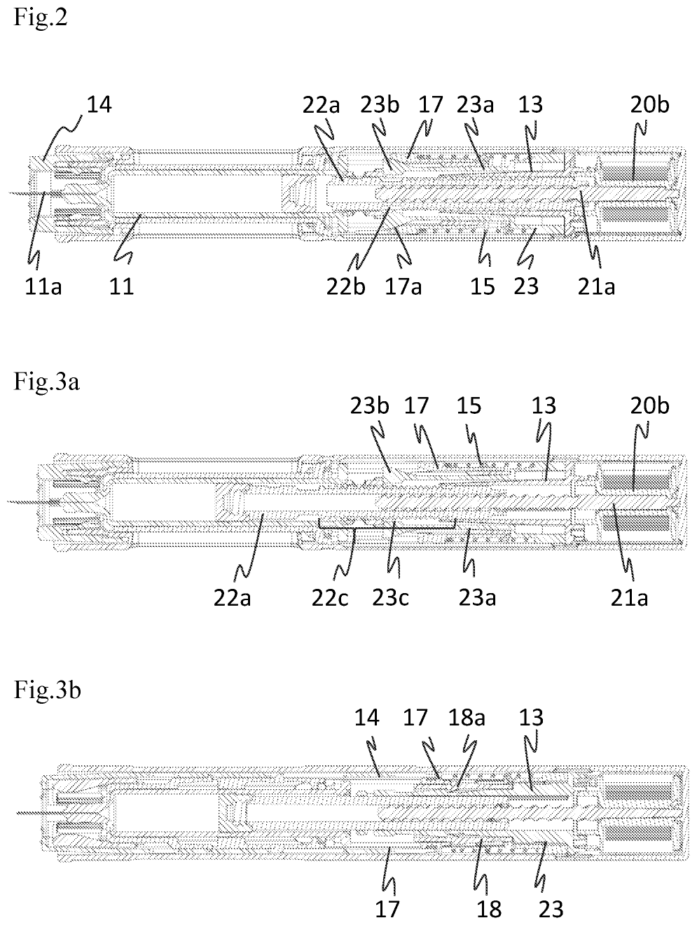

FIG. 2 shows a longitudinal section through the autoinjector at the moment of the triggering of the discharge. The needle-guard sleeve 14 is displaced in the proximal direction by contact with the injection site, and thus also the switching sleeve 17, the inner circumference of which has previously prevented the cams 23b from moving outward. Via control surfaces 17a, the switching sleeve 17 slides along the outer sides of the cams 23b in the proximal direction and releases the cams for a radial outward movement. As a result, the initial blockade of the propelling sleeve 22a is released, the axial force exerted by the discharge spring on the propelling sleeve 22a pushes the cams 23b out of the recesses 22b and the discharge starts.

FIG. 3a and FIG. 3b show two longitudinal sections rotated by 90° with respect to one another about the longitudinal axis through the autoinjector during discharging, wherein the upper section corresponds to that of FIG. 2. The needle-guard sleeve 14 is located in its proximal intermediate position and the switching sleeve 17 in a position proximal to the cams 23b. The engagement elements 23c slide over the latching elements 22c of a grid and can generate clicking noises which signal the progress of the discharging to the user. The locking sleeve 18 is located in its proximal end position and the inwardly moved locking members 18a engage behind edges of the mechanism holder 13 and can thus no longer be moved distally.

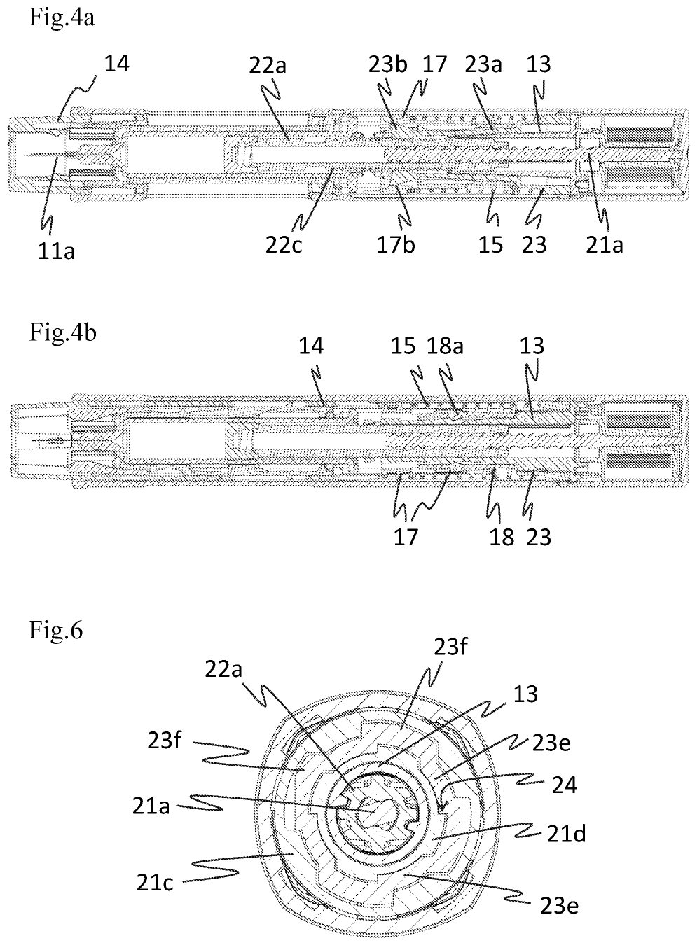

FIG. 4a and FIG. 4b show the two longitudinal sections from FIG. 3a and FIG. 3b in the blocking and locking states. After removal of the autoinjector from the injection site before the end of the discharging, the needle-guard spring 15 pushes the switching sleeve 17 as a control element according to the invention and the needle-guard sleeve 14 in the distal direction. In this case, each control cam 17a of the switching sleeve 17 slides over the outer side of a cam 23b and pushes the engagement element 23c into engagement with the corresponding grid of the propelling sleeve 22a. The axial movement of the propelling sleeve 22a is thereby interrupted. Locking surfaces 17b adjoining the control cams 17a in the proximal direction keep the engagement elements 23c engaged and block a further axial movement of the propelling sleeve 22a (FIG. 4a). As a result of the distal movement of the switching sleeve 17 relative to the locking sleeve 18, the locking members 18c snap behind a respective proximally directed edge of the switching sleeve 17 and lock the switching and needle-guard sleeve against being moved again in the proximal direction (FIG. 4b).

FIG. 5a and FIG. 5b show two longitudinal sections of a second embodiment of the autoinjector, which are rotated by 90° with respect to one another about the longitudinal axis, in the delivery state with the needle-guard cap 11b and the device cap 16 mounted thereon. The main differences from the first embodiment are explained below. A blocking unit 23 has a sleeve-shaped base with two flexible arms 23a mounted in the distal direction, at each end of which there is a cam 23b. An inner side of the cams 23b is adapted to a recess 13a of the axially fixed mechanism holder 13 and is held in an initial engagement with the recess 13a by the locking sleeve 18 in the delivery state of the autoinjector. This prevents an axial movement of the blocking unit 23. The blocking unit 23 comprises two rigid arms 23d extending in the proximal direction from the sleeve-shaped base. An axial coupling spring 25 in the form of a metallic coil spring is provided between a proximally directed surface of the rigid arms 23d and an axially fixed stop. The coupling spring 25 exerts a force on the blocking unit in the distal direction. The needle-guard spring 15 is mounted between the switching sleeve 17 and radial webs of the axially fixed mechanism holder 13, wherein the webs fix the mechanism holder 13 in the housing. The rigid arms 23d of the blocking unit penetrate or intersect the radial webs in the axial direction and are in turn connected to one another proximally to the webs to form a coupling sleeve. The spring coil 21b has a distal extension 21d comprising a coil sleeve arranged concentrically to the spring shaft with radially outwardly directed locking surfaces.

FIG. 6 shows a cross section through the autoinjector axially at the level of a proximal end of the rigid arms 23d of the blocking unit before discharge, indicated in FIG. 5 by an interrupted vertical line. At this point, the coupling sleeve of the blocking unit comprises four projections 23e, each offset by 90° and directed inward, as the first coupling element, which projections engage in a rotationally fixed manner via radial coupling surfaces 24 in four corresponding recesses on the distal widening 21d of the spring coil as the second coupling element. At the same time, four formations 23f of the coupling sleeve, which are distributed uniformly over the circumference and are directed outward, engage in four recesses of a spring sleeve 21c mounted in a rotationally fixed manner in the housing. The projections 23e and formations 23f have an angular extension of approximately 45°, and the inwardly directed projections are, moreover, offset against the outwardly directed formations, resulting in a more or less constant thickness of the coupling sleeve. As can be seen in FIG. 6, the projections/formations of the coupling sleeve arranged alternately inside and outside might also be referred to instead as recesses/indentations of the coupling sleeve arranged on alternate sides. The spring coil 21b is coupled to the housing 10b in a rotationally fixed manner by the two engagements, so neither the threaded rod 21a nor the propelling sleeve 22a moves.

FIG. 7a shows the longitudinal section from FIG. 5a in the released state. During the piercing operation, the locking sleeve 18 is moved away from the position of the recesses 13a by a first partial stroke of the proximal piercing movement of the needle-guard sleeve 14 and of the switching sleeve 17, so that the holding arms 23a can disengage radially and release the blocking unit 23. During a second partial stroke of the proximal piercing movement, a proximal end of the switching sleeve 17 hits a distally directed stop surface of the blocking unit 23, as indicated in FIG. 7a by two dotted arrows. As a result, the switching sleeve 17 pushes the blocking unit 23 in the proximal direction by a coupling stroke and the coupling spring 25 is tensioned. Because the coupling surfaces 24 of the inner projections 23e of the coupling sleeve and of the recesses of the widening 21d of the spring coil 21b have, respectively, an axial extension or an overlap of less than the coupling stroke, the engagements of the projections of the coupling sleeve with the recesses of the widening are thereby released, and the spring coil starts to rotate under the effect of the torsion spring. The coupling surfaces 24 of the widening 21d of the spring coil are located on indentations of a coil flange which is at a distance equal to at least the coupling stroke from the distal spring flange in the distal direction, or on formations on a coil sleeve which are at a distance equal to at least the coupling stroke from the distal spring flange in the distal direction.

FIG. 7b shows the longitudinal section from FIG. 5a in the blocked state after removal of the autoinjector from the puncture site. The needle-guard sleeve 14, which is moved in a securing movement from a rear end position into a front end position by a needle-guard spring, laterally covers the injection needle 11b. The switching sleeve 17 is also pushed distally by the needle-guard spring 15. The coupling spring 25 presses the blocking unit 23 distally to the extent of a coupling stroke, so that the coupling surfaces 24 of the inner projections 23e of the coupling sleeve and of the recesses of the extension 21d of the spring coil are brought into engagement again and block a rotation of the drive as in the initial state in FIG. 5. The discharging of any residual amount of liquid from the reservoir is thereby prevented. As in the first variant, a movement of the locking sleeve 17 in the distal direction is arrested and a movement of the switching sleeve 18 in the proximal direction is prevented by locking members of the locking sleeve 17.

The projections 23e which can be seen in FIG. 6 can also be formed directly on the rigid arms 23d of the blocking unit without these being connected to one another again to form a coupling sleeve. A guiding of the rigid arms through the webs of the mechanism holder can conduct the torque of the spring coil transmitted to the rigid arms via the locking surfaces to the housing; thus the formations 23f of the coupling sleeve can be dispensed with. The projections 23e can engage radially outward into inwardly directed recesses of an outer extension 21d of the spring coil.

The inner and the outer formations of the coupling sleeve and of their respective counterparts can differ in design, number, and/or axial arrangement. For example, the formations may assume the shape of axial ribs, and the recesses on the spring coil or spring sleeve may accordingly assume the shape of axial slots, or both formations and recesses are formed as teeth. The recesses on the spring sleeve may also be attached directly to the housing; the corresponding connection may, but does not have to be released during the coupling stroke. In view of the one-time use of the autoinjector and of the rotation blocking, the inner and outer formations of the coupling sleeve may also each be designed differently from one another as long as only the axial extension and arrangement of the inner projections allows release of the engagement by a coupling stroke and the outer formations are compatible with the rotational alignment of the holding arms of the coupling sleeve.

As in the first embodiment, the cams 23b on the flexible arms 23a of the blocking unit can also engage in recesses of the propelling sleeve and additionally secure them against an axial movement both in the delivery state and in the blocking state. As in the first variant, radially elastically mounted teeth on said cams can serve, in conjunction with a grid of the propelling sleeve, to generate clicking noises during the discharging. Alternatively, preferably axially elastically mounted teeth are conceivable on or in engagement with the extension 21d of the spring coil.

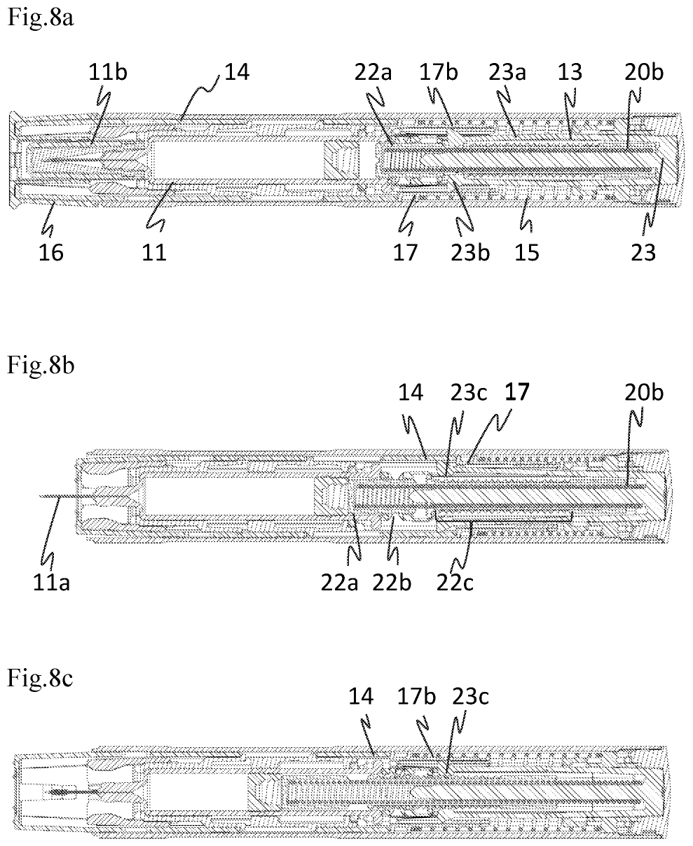

FIGS. 8a to 8c show a third embodiment in longitudinal section in the delivery state (FIG. 8a), at the moment of triggering (FIG. 8b), and in the blocking or locking state (FIG. 8c). The needle-guard cap 11b and the device cap 16 are shown in the delivery state. In contrast to the first exemplary embodiment, the autoinjector comprises a drive with a compression spring 20b designed as a coil spring, which is arranged at least partially within the propelling sleeve 22a and acts directly thereon. The blocking unit 23 comprises a proximal base from which two arms 23a, as a flexible mounting of the cams 23b and additionally a central pin within the compression spring, point in the distal direction. In turn, the switching sleeve 17 takes on the function of the control element and for this purpose forms the control surfaces and the locking surfaces 17b, by means of which the cams 23b are initially held in engagement with the recesses 22b of the propelling sleeve, or the engagement elements 23c are blocked in engagement with the latching elements when the discharging is interrupted. The propelling sleeve 22a also has a grid with latching elements 22c for engagement with engagement elements 23c on the inner sides of the cams 23b.

In the first and the third embodiment, the switching sleeve 17 takes on the function of the control element. Because the locking sleeve 18 is arranged coaxially within and practically at the same axial position as the switching sleeve 17 in the initial or delivery state, the locking sleeve preferably has two slots or axial recesses through which the control cams 17a can come into contact with the cams 23b. The embodiments shown can be combined with a mechanical or electronic end-clicking, delayed where appropriate, which indicates to the user the end of the injection and possibly a holding time. The engagement elements and the cams can also be arranged independently of one another, for example on different arms which are offset by 90° with respect to one another about the longitudinal axis. The needle-guard sleeve acts at least as a triggering or actuating sleeve with an initial position that is different from the final needle-guard position and from which the needle-guard sleeve is displaced into the proximal intermediate position, thereby triggering the discharging as well as a syringe movement in the distal direction.

FIG. 9a and FIG. 9b show two partial longitudinal sections of a fourth embodiment of the invention rotated by 90° with respect to one another about the longitudinal axis, similar to the second embodiment of FIGS. 5 to 7, in the delivery state. In contrast thereto, the force of the needle-guard spring serves in this case to push projections or engagement elements into a position blocking the drive element; a separate coupling spring can be dispensed with accordingly. In further contrast to the second embodiment, the axial blocking or coupling stroke of the blocking unit is not executed in the direction opposite a decoupling or triggering stroke, but in the same, proximal direction.

As in the first and second exemplary embodiments, a spiral spring 20a is anchored with its outer end in a rotationally fixed manner on a spring sleeve 21c, which spring sleeve in turn is accommodated in a rotationally fixed manner within the housing 10a as an independent component or else is part of a mechanism holder 13 fixedly anchored to the housing. The inner end of the coil spring 20a is connected in a rotationally fixed manner to a spring coil 21b, which rotates a rotation member in the form of a threaded rod 21a for discharging. The threaded rod 21a is coupled to the spring coil 21b in a rotationally fixed manner or even integrally formed therewith. A propelling element in the form of a propelling sleeve 22a has a threaded element on an inner side at a proximal end for engaging in an external thread of the threaded rod 21a. The propelling sleeve 22a is secured in a mechanism holder 13 against rotation by an axial groove or another deviation from a rotationally symmetrical outer side, so that the rotation produced by the rotation spring 20a is converted into a linear propelling movement.

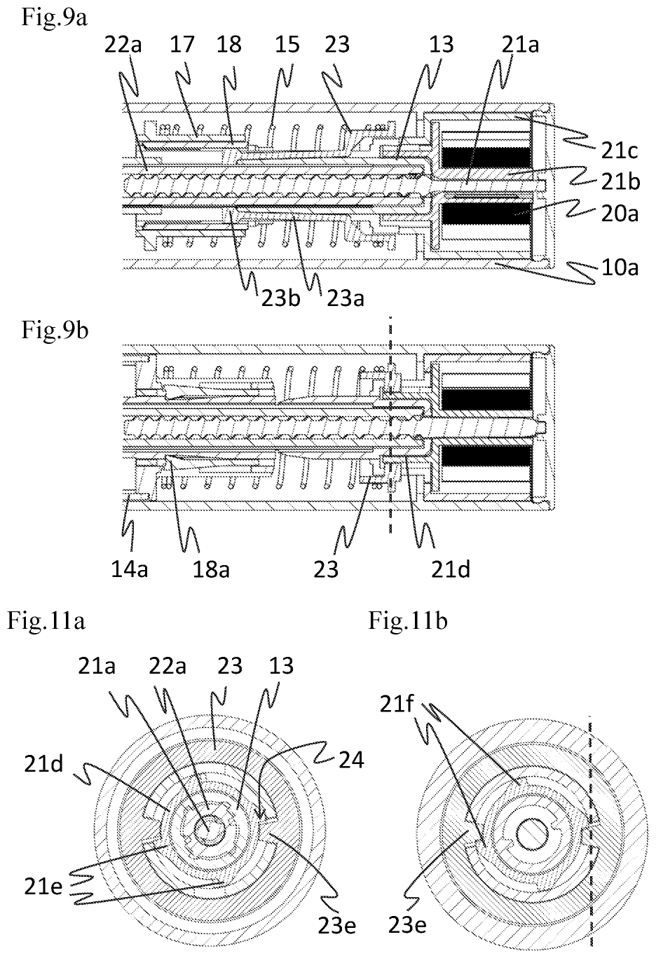

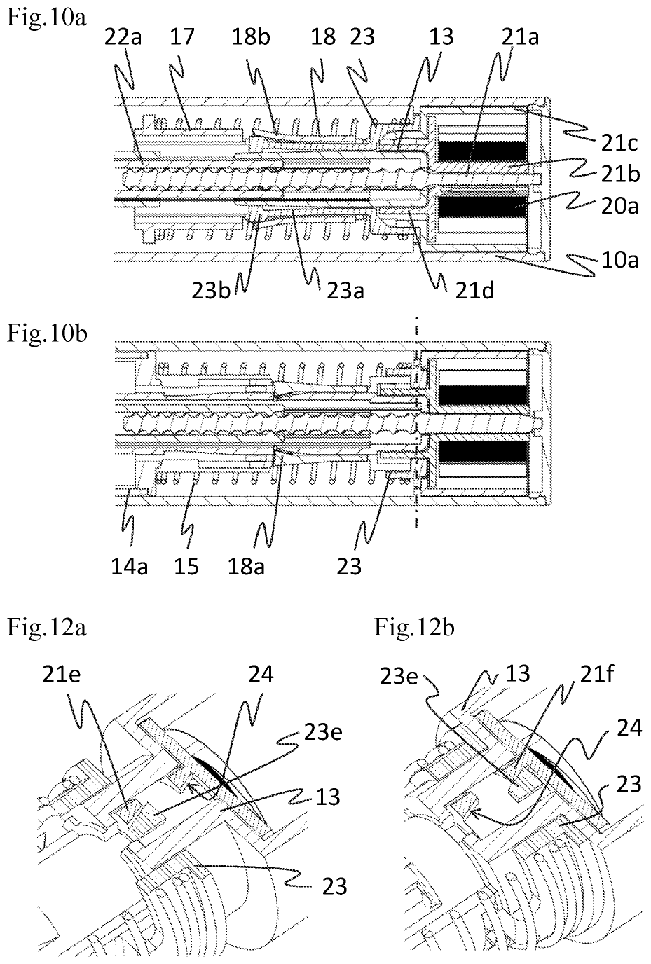

When the injection needle is inserted into the injection site along the longitudinal axis L, a needle-guard sleeve with two sleeve arms 14a is pushed in the proximal direction by an actuation stroke and against the force of a needle-guard spring 15 and thereby triggers a product discharge. After the injection has taken place or when the autoinjector is prematurely removed from the injection site, the needle-guard sleeve can be displaced by the needle-guard spring 15 relative to the housing 10a from an intermediate position along the longitudinal axis L in the distal direction into a needle-guard position and can be locked there against being pushed back again. The needle-guard spring 15 is a spring made of metal which acts as a compression spring and is designed as a coil spring and is supported at its proximal end on a blocking unit 23 or coupling sleeve. The blocking unit 23 has an annular proximal base with two flexible arms 23a mounted in the distal direction, at each end of which there is a cam 23b. An inner side of the cams 23b is adapted to a recess in the mechanism holder 13 and is prevented from radial deflection by a locking sleeve 18 in the delivery state. As a result of the actuating movement, the locking sleeve 18 is pushed in the proximal direction and an outer side of the cams 23b can spring radially outward next to distal edges of two portions 18b (FIG. 10a) of the locking sleeve 18. The blocking unit 23 is pushed in the proximal direction by the needle-guard spring 15, and the cams 23b, due to the engagement at the distal edges, in turn move the locking sleeve 18 further in the proximal direction up to a stop of the locking sleeve 18 in a proximal end position in abutment with the mechanism holder 13.

A switching sleeve 17 is arranged between a proximal end of the sleeve arms 14a and the needle-guard spring 15 and is at least partially surrounded by the needle-guard spring 15. The switching sleeve 17 is preferably snap-fitted or even integrally formed with the proximal end of the sleeve arms 14a. The locking sleeve 18 is positioned within and coaxially with respect to the switching sleeve 17, with two saw-tooth-shaped locking members 18a arranged offset by 180° about the longitudinal axis, each resiliently mounted on a spring arm which points in the distal direction. The locking sleeve 18 is coupled to the switching sleeve 17 via the locking members 18a in such a way that an actuating movement of the needle-guard sleeve 14 and the switching sleeve 17 also moves the locking sleeve 18 in the proximal direction. In a proximal end position of the locking sleeve 18, the locking members 18a are released from the switching sleeve 17 for an inward movement into corresponding recesses of the mechanism holder 13. Due to the spring action of the spring arms, the locking members 18a each engage behind a proximally directed edge of the mechanism holder 13 and thereby arrest the locking sleeve 18 against a distal movement. When the autoinjector is removed from the puncture site, the switching sleeve 17 is pushed by the needle-guard spring 15 in the distal direction over the locking members 18a, whereupon, as a result of the spring effect of the spring arms, the locking members each engage behind a proximally directed edge of the switching sleeve 17 in a locking position and lock the switching sleeve and the needle-guard sleeve against renewed movement in the proximal direction.

FIG. 10a and FIG. 10b show the longitudinal sections of FIG. 9a and FIG. 9b in the blocked state after removal of the autoinjector from the puncture site. By means of the needle-guard spring 15, the switching sleeve 17 and the needle-guard sleeve are pushed in the distal direction again, while the locking sleeve 18 is arrested by the locking members 18a against a distal movement, and locks a movement of the switching sleeve 17 in the proximal direction, as mentioned. In the distal end position of the switching sleeve 17, two radially flexible portions 18b of the locking sleeve 18 which are pointing in the distal direction and are arranged offset by 180° about the longitudinal axis and are aligned with the flexible arms 23a of the blocking unit 23, are released for radial deflection to the outside. The cams 23b, which are pushed in the proximal direction by the spring force of the needle-guard spring 15, spread the portions 18b out radially, as a result of which the cams 23b and the blocking unit 23 can slide under the portions 18b in the proximal direction. The distally directed edges of the portions 18b form an additional or alternative locking means for the switching sleeve 17. In contrast to the second exemplary embodiment, the blocking or coupling stroke of the blocking unit 23 is directed proximally and thus in the same direction as the triggering stroke, and causes coupling surfaces of the blocking unit and of the distal extension 21d of the spring coil 21b to engage, as a result of which the rotation of the spring coil 21b and the propelling of the propelling element 22a are blocked.

FIG. 11a shows a cross section through the autoinjector in the delivery state, axially at the height indicated in FIG. 9b by an interrupted vertical line. The blocking unit 23 comprises two projections 23e as first coupling elements, which elements are offset by 180° and are directed radially inward from the annular base, and which, as second coupling elements on a distal extension 21d of the spring coil, are in friction-locking contact with locking cams 21e via axis-parallel coupling surfaces 24. The projections 23e absorb the torque of the spring coil and directly lock a rotation of the threaded rod 21a. The engagement is released by an axial triggering stroke of the blocking unit 23 corresponding to at least the axial extension of the coupling surfaces, and the spring coil begins to rotate under the action of the torsion spring. Four locking cams 21e arranged 90° offset from one another are shown on the extension 21d. As a result, the initial positioning of the threaded rod 21a and thus of the propelling sleeve 22a can be set in smaller steps than with an identical number of projections 23e and locking cams 21e.

FIG. 11b shows a cross section through the autoinjector in the blocking state, axially at the height indicated in FIG. 10b by a dash-dotted vertical line. The projections 23e engage as engagement elements or third coupling elements in a friction-locking manner via coupling surfaces 24 in latching elements 21f as blocking cams or fourth coupling elements on a distal extension of the spring coil. The illustrated four latching elements 21f form a circular grid, wherein six, eight or even more latching elements can also be arranged distributed over the circumference in order to ensure a minimally delayed blocking. In the present case, the projections 23e or the coupling surfaces 24 serve simultaneously as first and third coupling elements, wherein the second and fourth coupling elements are offset differently and axially to the extent of the sum of the triggering and coupling stroke. Alternatively, two spaced-apart coupling elements could also interact on the blocking unit with the same coupling element on the spring coil. The illustrated blocking state is assumed when the discharge is interrupted, i.e., when the autoinjector is lifted off the injection site prematurely and, depending on the rotational orientation of the coupling elements, even after the autoinjector has been lifted off the skin at the regular end of the discharging, when the piston is present at the distal end of the syringe body.

FIG. 12a and FIG. 12b show on the left a longitudinal section through the autoinjector in the plane indicated in FIG. 11b by a broken vertical line parallel to and spaced apart from the longitudinal axis, and axially in the region of the cross sections from FIG. 11a and FIG. 11b in the delivery state (FIG. 12a) and in the blocking state (FIG. 12b). The coupling surfaces 24 between the projection 23e and the locking cams 21e, on the one hand, and between the projection 23e and the latching element 21f, on the other hand, are not formed parallel or perpendicular to the longitudinal axis, but in each case in the form of oblique, gear-like guide surfaces. As a result, a torque of the spring coil generates an axial, proximally directed force on the projection 23e, which supports both the triggering stroke and the coupling stroke.

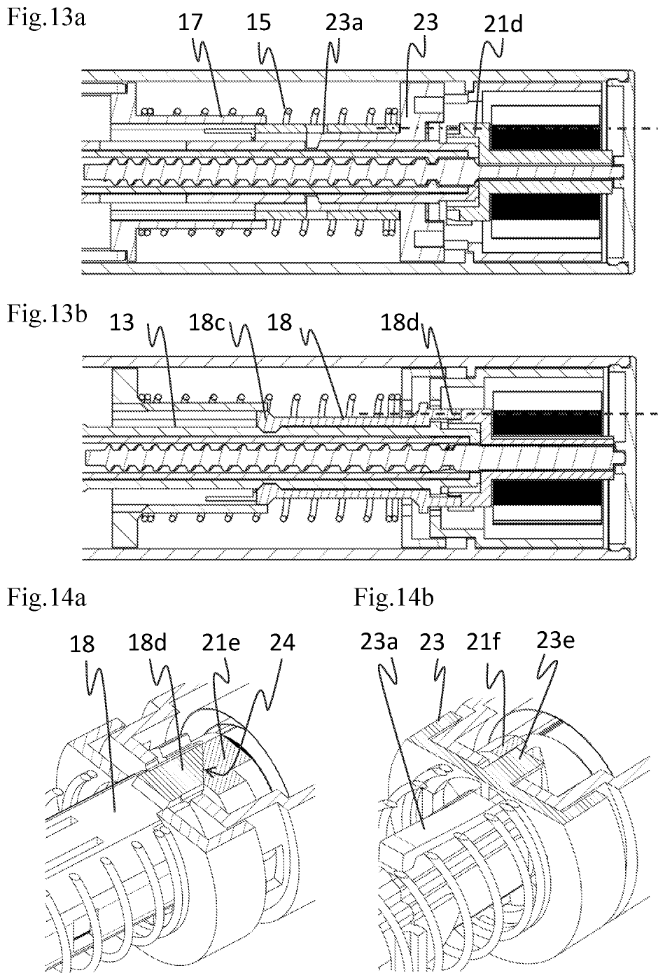

FIG. 13a and FIG. 13b show two partial longitudinal sections of a fifth embodiment of the invention which are rotated by 90° with respect to one another about the longitudinal axis, in a manner similar to the fourth embodiment, in the delivery state. In contrast thereto, the locking sleeve 18 serves here for the initial locking of the rotation of the spring coil 21d. For this purpose, the locking sleeve 18 is locked in a proximal position by locking cams 18c in recesses on the mechanism holder 13. At the proximal end, the locking sleeve 18 has projections 18d for the initial rotational blocking of the spring coil 21d. The needle-guard spring 15 is provided between the switching sleeve 17 and the blocking unit 23. The blocking unit 23 is locked against a movement in the proximal direction with arms 23a that are directed distally and are likewise held by the locking sleeve 18 in recesses of the mechanism holder 13.

FIG. 14a shows a longitudinal section through the autoinjector in the plane indicated in FIG. 13b by an interrupted horizontal line parallel to the longitudinal axis, in the delivery state. FIG. 14b shows a longitudinal section through the autoinjector in the plane indicated in FIG. 13a by a dash-dotted horizontal line parallel to the longitudinal axis, in the blocking state. The two sectional planes in FIG. 14a and FIG. 14b are therefore offset by 90° relative to one another. The coupling surfaces 24 between projection 18d as the first coupling element and locking cam 21e as the second coupling element are not formed parallel or perpendicular to the longitudinal axis, but in the form of oblique, gear-like guide surfaces. In the blocking state, a projection 23e of the blocking unit 23 is engaged with a latching element 21f of the spring coil.

Due to the actuating movement of the switching sleeve 17, the locking cams 18c are released to the outside for a radial movement. Via the oblique coupling surfaces 24, an axial, distally directed force is exerted on the projection 18d by the torque of the spring coil, whereby the locking sleeve 18 moves distally and enables the rotation of the spring coil 21d and thus the propelling of the propelling element. As a result of this initial displacement of the locking sleeve 18, it snaps together with the switching sleeve 17 and is moved distally by the switching sleeve when the autoinjector is removed from the injection site. In a distal end position, the locking sleeve snaps together with a component fixed to the housing and locks the switching sleeve 17 and the needle-guard sleeve against re-insertion.

In the case of an injection interruption, the rotational movement of the spring coil is stopped by the projection 23e of the blocking unit 23. Before and during the injection, the blocking unit 23 is axially blocked by the locking sleeve 18. When the autoinjector is lifted off the skin and the needle sleeve, switching sleeve 17, and locking sleeve 18 are thereby pushed axially in the distal direction, the blocking unit 23 is released and pushed axially in the proximal direction by the force of the needle-guard spring 15, whereby the projection 23e is coupled into the grid with the latching elements 21f and stops the rotational movement. The coupling surfaces between projection 23e as the third coupling element and latching element 21f as a fourth coupling element are oriented parallel to the longitudinal axis, but can also be designed in the form of oblique, gear-like guide surfaces.

### LIST OF REFERENCE SIGNS

10a housing part
10b closure cap
10c recess
11 prefilled syringe
11a injection needle
11b needle-guard cap
12 syringe holder
13 mechanism holder
13a holder recess
14 needle-guard sleeve
14a sleeve arm
15 needle-guard spring
16 device cap
17 switching sleeve
17a control cam
17b locking surface
18 locking sleeve
18a locking member
18b portion
18c locking cam
18d projection
20a coil spring
20b compression spring
21a threaded rod
21b spring coil
21c spring sleeve
21d coil extension
21e locking cam
21f latching element
22a propelling sleeve
22b sleeve recess
22c linear latching element
23 blocking unit
23a flexible arm
23b cam
23c radial engagement element
23d rigid arm
23e axial engagement element
23f formation
24 coupling surface
25 coupling spring

1. An autoinjector, comprising
a housing;
a propelling member;
a drive configured to move the propelling member in a discharge movement in a longitudinal direction relative to the housing for automatically discharging a liquid product contained in a product container held in the housing in an axially fixed manner;
a needle-guard spring for pretensioning a needle-guard sleeve in a distal direction, the needle-guard sleeve configured to move in a proximal direction in an actuating movement when the autoinjector is pressed against an injection site, and to move in the distal direction in a needle-guard movement when the autoinjector is removed from the injection site;
a grid comprising a multiplicity of latching elements; and
an engagement element which, by an engagement in the grid, is able to block a discharging movement of the propelling member,
wherein when the autoinjector is removed from the injection site without completely discharging the liquid product, the engagement element is pushed into the engagement with at least one of the multiplicity of latching elements by a pretensioned spring.

2. The autoinjector according to claim 1, wherein the drive comprises a rotating drive element, wherein the multiplicity of latching elements are arranged on the rotating drive element, and wherein the engagement element directly blocks the drive element in an axial coupling stroke.

3. The autoinjector according to claim 2, wherein the axial coupling stroke of the engagement element is released by the needle-guard movement of the needle-guard sleeve.

4. The autoinjector according to claim 3, wherein during the axial coupling stroke, the engagement element is moved in the proximal direction by the needle-guard spring.

5. The autoinjector according to claim 4, wherein the multiplicity of latching elements and the engagement element comprise corresponding coupling surfaces, the coupling surfaces formed obliquely relative to the longitudinal direction such that a force can be exerted on the engagement element in the proximal direction by a torque of the drive element.

6. The autoinjector according to claim 3, wherein the engagement element is a part of a coupling, and as a result of the actuating movement of the needle-guard sleeve, directly releases the drive element for rotation.

7. The autoinjector according to claim 6, wherein the coupling comprises a first coupling element, which can be removed, by an axial triggering stroke, from a second coupling element for triggering the rotation, and
wherein the coupling comprises a third coupling element, which can engage, by the axial coupling stroke and via a coupling surface, in a fourth coupling element for blocking the rotation of the drive element, the fourth coupling element including at least one of the multiplicity of latching elements.

8. The autoinjector according to claim 7, wherein the first and third coupling elements and the second and fourth coupling elements are identical.

9. The autoinjector according to claim 7, wherein the first and third coupling elements are identical, and the second and fourth coupling elements are axially spaced apart by a sum of the axial triggering stroke and the axial coupling stroke.

10. The autoinjector according to claim 3, wherein a first coupling element is removable by a distal triggering stroke from a second coupling element for triggering a rotation of the drive element, and a third coupling element can engage in a fourth coupling element for blocking the rotation of the drive element by a proximal coupling stroke.

11. The autoinjector according to claim 3, wherein during the axial coupling stroke, the engagement element is moved distally by a coupling spring.

12. The autoinjector according to claim 1, wherein the multiplicity of latching elements are arranged in the longitudinal direction, wherein a control element interacts with the engagement element via a control cam in such a way that, when the control element, driven by the needle-guard spring, is moved in the distal direction, the engagement element is brought into the engagement with at least one of the multiplicity of latching elements of the grid.

13. The autoinjector according to claim 12, further comprising a locking element with a locking surface configured to secure the engagement of the engagement element in a locking position held by the needle-guard spring.

14. The autoinjector according to claim 13, wherein the locking member is non-releasably held in the locking position.

15. The autoinjector according to claim 14, wherein locking element is coupled in the proximal direction in the locking position with the needle-guard sleeve locked in a needle-guard position, and wherein the control element and/or the locking element serves as a switching sleeve which is coupled axially to the needle-guard sleeve.

16. An autoinjector, comprising:
a housing;
a propelling member;
a drive configured to move the propelling member in a discharge movement in a longitudinal direction relative to the housing for automatically discharging a liquid product contained in a product container held in the housing in an axially fixed manner;
a needle-guard spring for pretensioning a needle-guard sleeve in a distal direction, the needle-guard sleeve configured to move in a proximal direction in an actuating movement when the autoinjector is pressed against an injection site, and to move in the distal direction in a needle-guard movement when the autoinjector is removed from the injection site; and
a coupling comprising an engagement element and a multiplicity of latching elements, wherein the engagement element is able to block the discharge movement of the propelling member by an engagement in at least one of the multiplicity of latching elements, and
wherein, when the autoinjector is removed from the injection site without completely discharging the product, the engagement element is pushed into the engagement with the at least one of the multiplicity of latching elements by a pretensioned spring.

17. The autoinjector according to claim 16, wherein the drive comprises a rotating drive element, wherein the multiplicity of latching elements are arranged on the rotating drive element, and wherein the engagement element directly blocks the drive element in an axial coupling stroke.

18. The autoinjector according to claim 17, wherein the axial coupling stroke of the engagement element is caused by the needle-guard movement of the needle-guard sleeve.

19. The autoinjector according to claim 18, wherein the coupling comprises a first coupling element and a second coupling element, wherein the first coupling element can be removed from the second coupling element to release the drive element for rotation by an axial triggering stroke caused by the actuating movement of the needle-guard sleeve.

20. The autoinjector according to claim 17, wherein during the axial coupling stroke, the engagement element is moved in the proximal direction by the needle-guard spring.
